(19) United States
(12) Patent Application Publication
(10) Pub. No.: US 2004/0220268 A1
(43) Pub. Date: Nov. 4, 2004

(54) Compound that directly stimulates phospholipase C activity

(76) Inventors: Yoe-Sik Bae, Busan (KR); Taohoon Lee, Pohang (KR); Pann-Ghill Suh, Pohang (KR); Sung Ho Ryu, Pohang (KR)

Correspondence Address: Joseph Hyosuk Kim, Ph.D., JHK Law, P.O. Box 1078, La Canada, CA, 91012-1078, US

(21) Appl. No.: 10/838,583

(22) Filed: May 3, 2004

Related U.S. Application Data
(60) Provisional application No. 60/467,607, filed on May 2, 2003.

(51) Int. Cl. A61K 31/195; A61K 31/18

(52) U.S. Cl. 514/562; 514/603

(57) ABSTRACT

The present application discloses a method for activating phospholipase C (PLC) activity in a cell by contacting a cell that contains phospholipase C with a compound of Formula I.

### CROSS-REFERENCE TO RELATED APPLICATIONS

The present application claims the benefit of priority to U.S. Provisional Application No. 60/467,607, filed May 2, 2003, which is incorporated by reference herein in its entirety.

### BACKGROUND OF THE INVENTION

1. Field of the Invention

The invention is directed to a method of using a compound of Formula I, including m-3M3FBS (2,4,6-Trimethyl-N-(meta 3-trifluoromethyl-phenyl)-benzenesulfonamide) and analogs and derivatives thereof for activating phospholipase C (PLC). The compound of Formula I, including m-3M3FBS may be used to stimulate phospholipase C activity, and increase intracellular levels of superoxide, calcium, and inositol phosphates. The invention also relates to using a control compound such as o-3M3FBS (2,4,6-Trimethyl-N-(ortho 3-trifluoromethyl-phenyl)-benzenesulfonamide) and analogs and derivatives thereof for use as an inactive analog.

2. General Background and State of the Art

Phosphoinositide (PI) hydrolysis is one of the important early signals associated with the stimulation of leukocytes by diverse extracellular stimuli (Rhee, 2001). Phospholipase C (PLC) hydrolyzes phosphatidylinositol 4,5-bisphosphates (PIP₂) into inositol-1,4,5-triphosphates (IP₃) and diacylglcerol (DAG), which mediate intracellular calcium release and the activation of protein kinase C (PKC), respectively (Rhee, 2001; Noh et al., 1995). Intracellular calcium ([Ca²⁺]ᵢ) increase and PKC activation subsequently induce diverse intracellular signalings, such as the activation of phospholipase A2, phospholipase D or mitogen-activated protein kinases. Finally, these intracellular signals result in the modulation of various cellular responses, including superoxide generation, secretion and proliferation in leukocytic cells (Bae et al., 1999; McLaughlin et al., 2001; Kim et al., 1999). Eleven isoforms of PLC are known (Rhee, 2001). While the β isoforms are known to be modulated by GTP-binding proteins, the γ isoforms have been reported to be activated by the stimulation of growth factor receptors (Rhee, 2001; Noh et al., 1995). Although many extracellular ligands that stimulate cell surface receptors leading to the activation of PLC β or γ have been reported, no direct PLC activity modulator has been identified until now.

Recently many synthetic compounds have been reported to modulate diverse immune responses (Tian et al., 1998; Zhang et al., 1999; Rosania et al., 1999). Synthetic compounds are known to regulate cellular activity by modulating cellular target proteins (Tian et al., 1998; Zhang et al., 1999; Rosania et al., 1999). While some of the compounds bind to cell surface receptors and induce receptor-mediated intracellular signals, others directly modulate intracellular target molecules after penetrating cells (Rosania et al., 1999; Strizki et al., 2001). The identification of compounds that modulate important physiological responses gives us two pieces of critical information: 1) They enable the development of synthetic compounds that modulate certain cellular functions, and 2) Their cellular target molecules can also be regarded as potential drug targets. In this respect, it is important not only to develop synthetic compounds that modulate cellular responses, but also to identify their target cellular proteins.

In this study, we screened a chemical library consisting of more than 10,000 different species in an effort to find a chemical that can stimulate superoxide generation in human neutrophils. We found that the compound, m-3M3FBS (2, 4, 6-Trimethyl-N-(meta 3-trifluoromethyl-phenyl)-benzenesulfonamide), can stimulate human neutrophils and that this leads to superoxide generation. By studying the action mechanism of m-3M3FBS, we suggest that the compound stimulates neutrophil activity by directly activating PLC.

### SUMMARY OF THE INVENTION

Superoxide anion, a potent oxidant, is a short-lived oxygen radical that is released into the extracellular environment of stimulated leukocytes (monocytes, macrophages, and polymorphonuclear leukocytes). Although excessive superoxide anions are harmful to normal cells, it may play an important role in bactericidal activity. For example, in a recent report (Wolach, B. et al. (1998) Blood Cells, Molecules, and Disease 24(23):544-551), a patient with idiopathic myelofibrosis whose superoxide anion generation was very low (28% of control) demonstrated significant bactericidal defect. In addition, superoxide anion release in 25% of patients with polycythemia Vera was below 60% of healthy control individuals Thus, effective control of superoxide anion generation in vivo has significant potential to treat infectious diseases because of its strong oxidative activity.

Phosphoinositide-specific phospholipase C (PLC) plays a pivotal role in the signal transduction of various cellular responses. However, although it is undeniably important that modulators of PLC activity be identified, no direct PLC activity modulator has been identified until now. In this study, by screening more than 10,000 different compounds in human neutrophils, we identified a compound that strongly enhances superoxide-generating activity, which is well known to be PLC-dependent. The active compound of Formula I, in particular 2, 4, 6-trimethyl-N-(meta 3-trifluoromethyl-phenyl)-benzenesulfonamide (m-3M3FBS) stimulated a transient intracellular calcium ([Ca²⁺]ᵢ) increase in neutrophils. Moreover, m-3M3FBS stimulated the formation of inositol phosphates in U937 cells, indicating that it stimulates PLC activity. The compound showed no cell type specificity in terms of [Ca²⁺]ᵢ increase in the various cell lines including leukocytes, fibroblasts, and neuronal cells. We also ruled out the possible involvement of heterotrimeric G-proteins in m-3M3FBS-stimulated signaling by confirming that: 1) pertussis toxin does not inhibit m-3M3FBS-induced [Ca²⁺]ᵢ increase; 2) m-3M3FBS does not stimulate cyclic AMP generation; 3) the inhibition of Gq by the regulator of G-protein signaling 2 does not affect the m-3M3FBS-induced [Ca²⁺]ᵢ increase. We also observed that m-3M3FBS stimulated PLC activity in vitro. The tested purified several isoforms of PLC (i.e., β2, β3, γ1, γ2, and δ1) were activated by m-3M3FBS and showed no isoform-specificity. Taken together, these results demonstrate that m-3M3FBS modulates neutrophil functions by directly activating PLC. As m-3M3FBS is the first compound known to directly activate PLC, it should prove useful for the study of the basic molecular mechanisms of PLC activation and PLC-mediated cell signaling.

The invention is directed to a method for activating phospholipase C (PLC) activity in a cell comprising contacting a cell comprising phospholipase C with compound of Formula I:

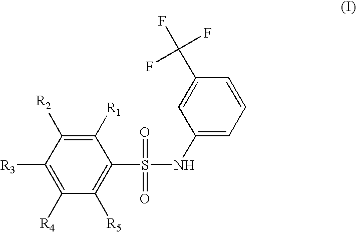

wherein

R₁ to R₅ are independently selected from H, OH group, a C₁-C₄ alkyl group, a halogen, an OR₆ group, COOH, COOR₇, an alkylene ester group according to the structure-(CH₂)ₘCOOR₇ or a CF₃ group;

R₆ is a C₁-C₄ alkyl group (preferably, C₁-C₄ alkyl), or a COR₇ group; R₇ is a C₁-C₂₀ alkyl group, preferably a C₁-C₄ alkyl group.

The compound of Formula I may be 4-tert-Butyl-N-(3-trifluoromethyl-phenyl)-benzenesulfonamide, 3,5-Di-tert-butyl-4-fluoro-N-(3-trifluoromethyl-phenyl)benzenesulfonamide, or 2,4,6-Trimethyl-N-(meta-3-trifluoromethyl-phenyl)-benzenesulfonamide (m-3M3FBS). In particular, the compound of Formula I may be 2,4,6-Trimethyl-N-(meta 3-trifluoromethyl-phenyl)-benzenesulfonamide (m-3M3FBS).

In the above-described method, the PLC may be isoform beta, gamma or delta. Further in the method, the compound of Formula I does not inhibit heterotrimeric G proteins, PI 3-kinase or phospholipase D. And in the method described above, the PLC activity is production of superoxide. The PLC activity may include increasing intracellular level of calcium or stimulating formation of inositol phosphate formation in a cell. The cell may be without limitation a neutrophil, neuronal cell or fibroblast.

In another aspect of the invention, in practicing the invention as described above, the method further comprises contacting the cell with an inactive control compound and comparing results of the compound of Formula I and the control compound. Such a control compound may be 2,4,6-Trimethyl-N-(ortho 3-trifluoromethyl-phenyl)-benzenesulfonamide (o-3M3FBS) or a derivative or analog thereof, which does not activate PLC.

In still another aspect, the invention is directed to a method for screening for candidate antitumor agent comprising contacting a cell expressing phospholipase C with the compound of Formula I to activate the phospholipase C activity, and then contacting the cell with a compound to determine whether phospholipase C activity is inhibited, wherein inhibition of phospholipase C activity indicates that the compound is an anti-tumor candidate compound.

In another aspect of the invention, the invention is directed to a method for treating a brain disorder in a subject comprising administering to a subject thereof therapeutically effective amount of the compound of Formula I.

The invention is also directed to instructions that sets forth the method of using the compound of Formula I as described above. The instructions may be in written form, and may take the form of a web page, catalog or an experimental manual.

The invention is also directed to a method for controlling PLC-mediated cell signaling comprising contacting a cell comprising phospholipase C with controlled amounts of the compound of Formula I.

Still further, the invention is directed to a method for increasing bactericidal activity in a subject comprising administering to the subject an effective amount of the compound of Formula I.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows effect of m-3M3FBS on superoxide anion generation in human neutrophils. Superoxide anion generation was measured by monitoring chemiluminescence in the presence of the chemiluminogenic probe lucigenin. Prepared neutrophils were stimulated with m-3M3FBS or o-3M3FBS at 2.5, 5, 10, 15, 20, 25, and 50 μM and lucigenin (40 μM) was then added. Luminescence changes were measured using a Luminoskan. Data are presented as means ±S. E. M. of four independent experiments (A). Structures of m-3M3FBS (2,4,6-Trimethyl-N-(meta 3-trifluoromethyl-phenyl)-benzenesulfonamide) and o-3M3FBS (2,4,6-Trimethyl-N-(ortho 3-trifluoromethyl-phenyl)-benzenesulfonamide) (B).

FIG. 2 shows effect of m-3M3FBS on [Ca²⁺]ᵢ and the inhibition of m-3M3FBS induced-calcium increase by U-73122. Fura-2-loaded neutrophils were stimulated with m-3M3FBS or o-3M3FBS at 25 μM in extracellular calcium containing 2 mM or extracellular free calcium conditions. The results shown are representative of three independent experiments (A). Prepared neutrophils were loaded with fura-2 and then stimulated with 25 μM of m-3M3FBS in the absence or in the presence of 4 μM of U-73122 or U-73343 (B). Changes in 340 nm/380 nm excitation ratio were monitored, and converted to [Ca²⁺]ᵢ levels. Data are presented as means ±S. E. M. of three independent experiments.

FIG. 3 shows effect of m-3M3FBS on in vivo PLC activity. U937 Cells were labeled with myo-[³H] inositol (1 μCi/10⁶ cells) for 24 h at 37° C. and then treated with various concentrations of m-3M3FBS or o-3M3FBS. Total inositol phosphates were eluted with a solution containing 1 M ammonium formate and 0.1 M formic acid. Radioactivity of the [3H] inositol phosphates was determined by counting in a scintillation counter. Data are presented as means ±S. E. M. of five independent experiments.

FIG. 4 shows effect of m-3M3FBS on [Ca²⁺]ᵢ in several cell lines. Neutrophils and HL60, and differentiated HL60, U937, NIH3T3, and PC12 cells were loaded with fura-2 and then stimulated with 25 μM of m-3M3FBS. Changes in the 340 nm/380 nm excitation ratio were monitored, and converted to [Ca²⁺]ᵢ levels. Data are presented as means ±S. E. M. of three independent experiments.

FIG. 5 describes cross-desensitization experiments. Fura-2-loaded neutrophils were pretreated with m-3M3FBS (25 μM) and then rechallenged with ATP (500 μM) or WKYMVm (1 μM) in this or in the reverse order in the extracellular calcium free condition. Changes in the 340 nm/380 nm excitation ratio were monitored, and converted to [Ca²⁺]ᵢ levels. The results shown are representative of three independent experiments.

FIG. 6 shows effect of m-3M3FBS on the activity of heterotrimeric G proteins. Isolated neutrophils were preincubated with PTX (1 μg/ml) or vehicle for 90 min at 37° C. and stimulated with m-3M3FBS, FMLF, or ATP at concentrations of 25, 1, and 500 μM, respectively. Changes in the excitation ratios at 340 nm/380 nm were monitored. Data are presented as means ±S. E. M. of two independent experiments (A). cAMP levels were determined by a radio receptor assay. Prepared cells were treated with m-3M3FBS (25 μM) or histamine (100 μM) and then lysed with Tris-EDTA. After removing the supernatant, [ 3H] cAMP and protein were added, and the amount of [³H] cAMP-protein complex was determined. The amount of unlabeled cAMP (in the sample) was then calculated by measuring the protein-bound radioactivity. Data are presented as means ±S. E. M. of two independent experiments (B). RGS2-GFP inducible HeLa cells were cultured for 48 h in the absence or in the presence of tetracycline, and the induction of RGS2-GFP was confirmed by Western blot analysis using anti-GFP antibody (C). RGS2-GFP inducible HeLa cells were cultured for 48 h in the absence or in the presence of tetracycline and loaded with fura-2. Cells were treated with histamine (100 μM) or m-3M3FBS (25 μM), and changes in the 340 nm/380 nm ratios were monitored and converted to [Ca²⁺]ᵢ values. The results shown are representative of three independent experiments (C).

FIG. 7 shows effect of m-3M3FBS on the activity of PLC isotypes in vitro. The hydrolyzing activity of PtdIns-4,5-P₂ was measured on mixed phospholipid vesicles containing 120 μM phosphatidylethanolamine, 30 μM PtdIns-4,5-P₂, and 1 μCi/ml [3H] PtdIns-4,5-P₂. Purified PLC β2, substrates, and the chemicals (2.5, 5, 10, 20, and 25 μM of m-3M3FBS or of o-3M3FBS) were coincubated (A), 25 μM of m-3M3FBS or o-3M3FBS, and phospholipid substrates were mixed with purified PLC β3, γ1, 2, or δ1 (B). Reactions were performed for 10 min at 37° C. in a 200 μl reaction mixture containing lipid micelles (5 μM [³H] PtdIns-4,5-P₂, 20,000 cpm). Reactions were stopped by adding 1 ml of CHCl₃:CH₃OH:HCl (50:50:0.3, v/v/v). Inositol trisphosphates were extracted with 0.5 ml of 1 M HCl/5 mM EGTA, and the radioactivity in the upper aqueous phase was measured. Data are presented as means ±S. E. M. of three independent experiments (A, B).

### DETAILED DESCRIPTION OF THE PREFERRED EMBODIMENTS

In the present application, “a” and “an” are used to refer to both single and a plurality of objects.

The present invention is directed to the use of a compound having formula I

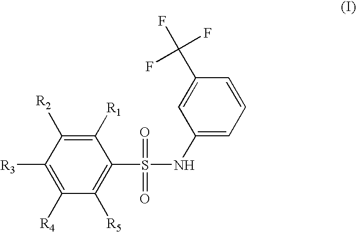

wherein

R₁ to R₅ are independently selected from H, OH group, a C₁-C₄ alkyl group including tert-butyl, a halogen such as Cl or F, an OR₆ group, COOH, COOR₇, an alkylene ester group according to the structure-(CH₂)ₘCOOR₇ or a CF₃ group;

R₆ is a C₁-C₄ alkyl group (preferably, C₁-C₄ alkyl), or a COR₇ group; R₇ is a C₁-C₂₀ alkyl group, preferably a C₁-C₄ alkyl group.

A preferred compound is:

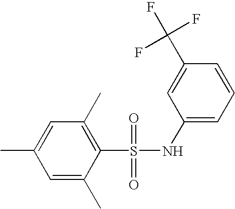

wherein, R₁, R₃, R₅ is CH₃.

Another preferred compound is:

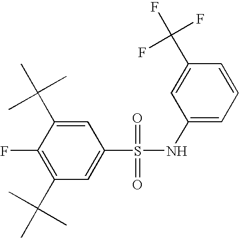

wherein, R₂, and R₄ is t-butyl, and R₃ is F.

Another preferred compound is:

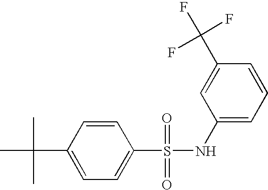

wherein R₃ is t-butyl.

We identified a synthetic compound of Formula I that stimulates superoxide generation in human neutrophils. In a particular embodiment, m-3M3FBS also evoked [Ca²⁺]ᵢ increases not only in neutrophils but also in several other cells, including neuronal cells and fibroblasts, and therefore, showed no cell type specificity. Through a study of its mode of action, we found that it directly activates many PLC isozymes, again without showing isozyme specificity.

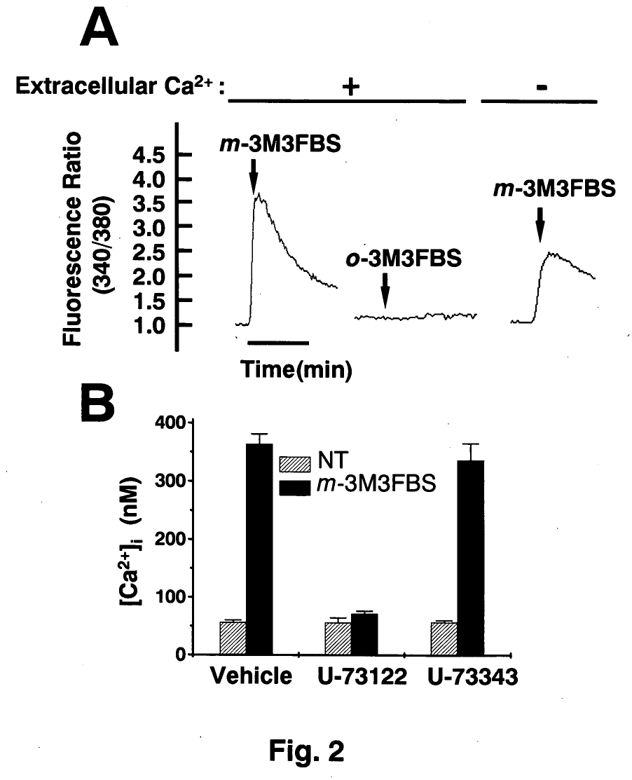

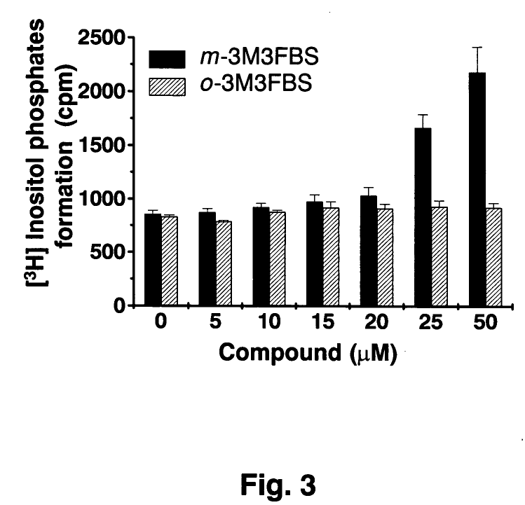

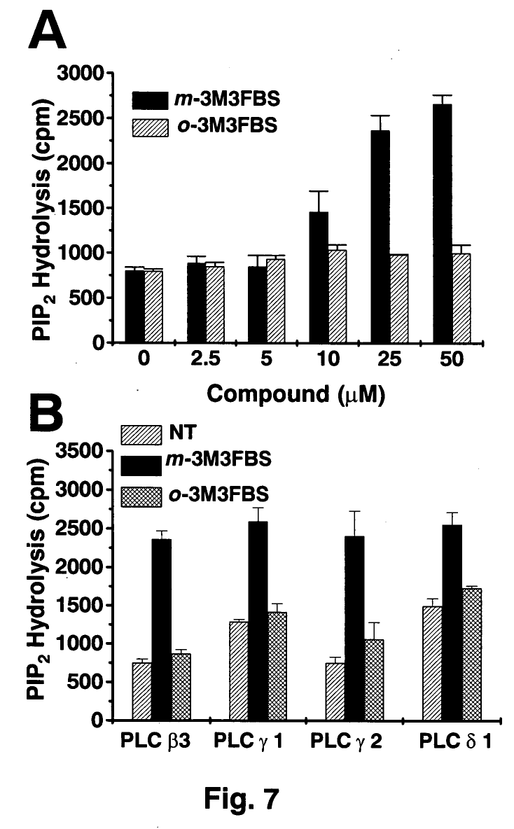

Our experiment demonstrates that stimulation of human neutrophils with a compound of Formula I, and in particular m-3M3FBS induces [Ca²⁺]ᵢ increases (FIG. 2A), and that this effect of m-3M3FBS is inhibited by U73122, a specific PI-PLC inhibitor (FIG. 2B). The stimulation of U937 cells with m-3M3FBS also caused PI hydrolysis (FIG. 3). During experiments designed to investigate the effect of m-3M3FBS on PLC activity, we observed that it activated several isoforms of PLC in vitro (FIGS. 7A and 7B). These results suggest that m-3M3FBS activates PLC directly ruling out a possible non-PLC dependent mechanism of the compound. Since all isoforms of PLC tested (β2, β3, γ1, γ2, and δ1) were activated by m-3M3FBS in vitro, it appears that the compound does not have any isoform-specificity in terms of the activation of this enzyme (FIG. 7B). Although many different extracellular ligands are known to stimulate PLC activity by binding to their specific cell surface receptors, there has been no report on the direct activation of PLC until now. Generally, the activation of cell surface receptors induces diverse signaling pathways. PLC activation is one of the earliest responses downstream of receptor stimulation (Rhee, 2001; Noh et al., 1995). Several previous reports have suggested that PLC is involved in several important cellular functions, such as, proliferation, differentiation, and apoptosis (Rhee, 2001; Noh et al., 1995; Rhee and Bae, 1997). However, the complications of cellular receptor-mediated signaling hinder our understanding of the nature of the signals and of the cellular responses regulated by PLC. Bearing this in mind, the identification of a molecule that can modulate PLC activity directly will undoubtedly be helpful for the elucidation of PLC-mediated cellular signaling and physiological responses. Furthermore, no ligand has been identified that stimulates the isoforms of PLC, including PLCδ1 and δ2. Since m-3M3FBS could stimulate PLCδ1 activity directly, it should be useful for the study of cellular signaling and functional events downstream of the enzyme.

In our in vitro experiments, we observed that m-3M3FBS stimulated the β, γ, and δ isoforms of PLC, and that it showed no isoform-specificity (FIGS. 7A and 7B). Moreover, the primary structures of the several different isoforms of PLC are known (Rhee, 2001; Noh et al., 1995). PLC β and PLC δ have an NH₂-terminal PH domain, an EF-hand, X and Y domains, known to form the catalytic core, and a COOH-terminal C2 domain. In addition, PLC β has a long C-terminal tail beyond the C2 domain (Rhee, 2001; Noh et al., 1995; Rhee and Bae, 1997). PLC γ has additional three SH domains between the X and Y domains, but no long COOH-terminal tail (Rhee, 2001; Noh et al., 1995; Rhee and Bae, 1997). Our study shows that m-3M3FBS stimulates three subfamilies of the PLC isoforms (PLC β, γ, and δ) (FIGS. 7A and 7B). This suggests that the compound acts on a common conserved region of these three isoforms, thus ruling out the possible involvement of the SH domains and COOH-terminal tail of PLC β. For the proper activation of PLC enzyme, calcium has been regarded as an essential requirement (Rhee and Bae, 1997). Calcium is required not only for the functioning of C2 domain that mediates the Ca²⁺-dependent binding to lipid vesicles but also for the catalytic activity of the enzyme (Rhee and Bae, 1997). In our study, we found that m-3M3FBS stimulated in vitro PLC activity in the presence or absence of Ca²⁺ (data not shown). The result suggests that m-3M3FBS may stimulate PLC activity with different mechanism from calcium ion. Previously, Carpenter et al., demonstrated that the addition of a purified X and Y domain in vitro showed lipase activity (Horstman et al., 1996). An investigation of the effect of m-3M3FBS on the lipase activity of an X and Y domain mixture will be required to confirm the possible action of m-3M3FBS on the two catalytic cores. Since m-3M3FBS is the first compound that directly stimulates PLC activity, the elucidation of the action mechanism of the compound will give useful information on the basic molecular mechanisms of the activation of PLC enzymes.

In FIG. 7, we demonstrated that all tested PLC isoforms were activated by m-3M3FBS. The result led us to check whether the compound act specifically on PLC or act on other enzymes that recognize phosphoinositols as substrates such as phosphoinositide-3-kinase (PI3-kinase). For this, we tested the effect of m-3M3FBS on the Akt phosphorylation that is dependent on the PI3-kinase in U937 cells. 50 μM of m-3M3FBS couldn't significantly increase the phosphorylation level of Akt (data not shown). It indicates that m-3M3FBS has specificity for PLC but not for PI3-kinase. To check the effect of m-3MFBS on other phospholipase, we also tested the effect of m-3M3FBS on the in vitro activity of phospholipase D. We observed that the compound did not affect on the activity of phospholipase D (data not shown). The result support our notion that m-3M3FBS acts specifically on PLC.

In conclusion, by screening a chemical library we identified a small synthetic molecule that potently stimulated superoxide generation. This is the first report of a direct activator of PLC, and the compound is useful for the study and control of PLC-mediated cell signaling.

Phospholipase C Activity in Tumorigenesis

Phospholipase C-gamma1 (PLC-gamma1) mediates signals from various extracellular origins to evoke cellular events such as mitogenesis. PLC-gamma1 is known to be highly expressed in colorectal cancer and familial adenomatous polyposis, indicating that PLC-gamma1 may be oncogenic. Further, rat fibroblasts that overexpress whole PLC-gamma1 show a transformed phenotype and are tumorigenic when transplanted into nude mice. These results indicate that overexpression of PLC-gamma1 could transform rat fibroblasts (Chang et al, Cancer Res. Dec. 15, 1997;57(24):5465-8).

Since the compound of Formula I activates phospholipase C activity, the inventive compound of Formula I may be used to screen for a candidate compound that inhibits the activation of phospholipase C activity. Such an inhibitory candidate compound may be useful as an anti-tumor agent. The screening method may comprise contacting a cell expressing phospholipase C with the compound of Formula I to activate the phospholipase C activity, and then at a suitable time, contacting the cell with a candidate compound to determine whether phospholipase C activity is inhibited. A control compound such as o-3M3FBS may be used in place of the compound of Formula I. Other control compounds may be used along with the candidate compound to compare and assess any inhibitory effective the candidate compound may have on phospholipase C activity.

Phospholipase C Activity in Proper Brain Function

A variety of extracellular signals are transduced across the cell membrane by the enzyme phosphoinositide-specific phospholipase C-beta (PLC-beta) coupled with guanine-nucleotide-binding G proteins. There are four isoenzymes of PLC-beta, beta1-beta4. Generation of null mutations in mice indicate that PLCbeta1−/− mice developed epilepsy and PLCbeta4−/− mice showed ataxia. PLC-beta1 is involved in signal transduction in the cerebral cortex and hippocampus by coupling predominantly to the muscarinic acetylcholine receptor, whereas PLC-beta4 works through the metabotropic glutamate receptor in the cerebellum, illustrating how PLC-beta isoenzymes are used to generate different functions in the brain (Kim et al., Nature, Sep. 18, 1997;389(6648):290-3).

The compound of Formula I activates phospholipase C. Since the lack of expression or activity of phospholipase C, such as PLC-beta1 to beta4, causes disorders in the brain such as epilepsy or ataxia, it is contemplated that the compound of Formula I may be administered to a subject suffering from a brain disorder due to a lack of phospholipase C activity to treat the symptoms of brain disorder such as epilepsy or ataxia in the subject.

Therapeutic Composition

In one embodiment, the present invention relates to treatment for various tumors or disorders of the brain that are characterized by either too much activation of an isozyme of phospholipase C (tumors) or too little activity of PLC (brain disorder). The inventive therapeutic compound may be administered to human patients who are either suffering from, or prone to suffer from the disease by providing compounds that either activate or inhibit the activation of PLC. In particular, the disease is associated with tumorigenesis, neurodegenerative disorder of the brain, loss of nerve cell, particularly in the hippocampus and cerebral cortex, cerebrovascular degeneration, and/or loss of cognitive ability. Further in particular, the present invention is directed to reducing tumors, and treating symptoms of epilepsy and ataxia.

The formulation of therapeutic compounds is generally known in the art and reference can conveniently be made to Remington's Pharmaceutical Sciences, 17th ed., Mack Publishing Co., Easton, Pa., USA. For example, from about 0.05 μg to about 20 mg per kilogram of body weight per day may be administered. Dosage regime may be adjusted to provide the optimum therapeutic response. For example, several divided doses may be administered daily or the dose may be proportionally reduced as indicated by the exigencies of the therapeutic situation. The active compound may be administered in a convenient manner such as by the oral, intravenous (where water soluble), intramuscular, subcutaneous, intra nasal, intradermal or suppository routes or implanting (eg using slow release molecules by the intraperitoneal route or by using cells e.g. monocytes or dendrite cells sensitised in vitro and adoptively transferred to the recipient). Depending on the route of administration, the peptide may be required to be coated in a material to protect it from the action of enzymes, acids and other natural conditions which may inactivate said ingredients.

For example, the low lipophilicity of the peptides will allow them to be destroyed in the gastrointestinal tract by enzymes capable of cleaving peptide bonds and in the stomach by acid hydrolysis. In order to administer peptides by other than parenteral administration, they will be coated by, or administered with, a material to prevent its inactivation. For example, peptides may be administered in an adjuvant, co-administered with enzyme inhibitors or in liposomes. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEP) and trasylol. Liposomes include water-in-oil-in-water CGF emulsions as well as conventional liposomes.

The active compounds may also be administered parenterally or intraperitoneally. Dispersions can also be prepared in glycerol liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, chlorobutanol, phenol, sorbic acid, theomersal and the like. In many cases, it will be preferable to include isotonic agents, for example, sugars or sodium chloride. Prolonged absorption of the injectable compositions can be brought about by the use in the composition of agents delaying absorption, for example, aluminium monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterile active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from a previously sterile-filtered solution thereof.

When the compounds are suitably protected as described above, the active compound may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations.

As used herein “pharmaceutically acceptable carrier and/or diluent” includes any and all solvents, dispersion media, coatings antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active compound is generally present in from about 0.5 μg/ml of carrier. In the case of compositions containing supplementary active ingredients, the dosages are determined by reference to the usual dose and manner of administration of the said ingredients.

Instructions

The present invention is also directed to instructions regarding the use of m-3M3FBS for stimulating phospholipase C activity, and increasing intracellular levels of superoxide, calcium, and inositol phosphate. Such instructions may be in a permanent or temporary format. The instructions may be in written form, such as but not limited to a textbook, protocol book, catalog, internet web site and so on. Such instructions may be in relation to but not limited to the sale and use of m-3M3FBS. The instructions may be presented via a computer screen on a cathode ray tube, LCD, LED, and so on, so long as the instructions are visible through the eye. The instructions may also be in the form of audio/visual media.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

Materials and Methods

Materials

Compounds were purchased from the Chembridge Corporation (San Diego, Calif.). Peripheral blood mononuclear cell (PBMC) separation medium (Histopaque-1077), dioleoyl-phosphatidylethanolamine, and tetracycline were purchased from Sigma (St. Louis, Mo.). RPMI 1640 and DMEM were from Life Technologies, Inc. (Grand Island, N.Y.), dialyzed fetal bovine serum and supplemented bovine calf serum from Hyclone Lab. Inc. (Logan, Utah), U-73122 and U-73343 from RBI (Natick, Mass.), hygromycin B and pertussis toxin (PTX) from Calbiochem (San Diego, Calif.), Myo-[2-³H] inositol (18.3Ci/mmol), [8-³H] Adenosine 3′,5′-cyclic phosphate, and Phosphatidylinositol-4,5-Bisphosphate (inositol-2-[³H]) from Amersham Corp. (Arlington Height, Ill.), and the AG 1-X 8 resin was purchased from Bio-Rad (Hercules, Calif.).

Methods

### Example 1

### Isolation of Human Neutrophils

Peripheral blood leukocytes were donated by the Ulsan Red Cross Blood Center (Ulsan, Korea). Human neutrophils were isolated by standard dextran sedimentation, the hypotonic lysis of erythrocytes, and by using a lymphocyte separation medium gradient, as described previously (Bae et al., 2001). Isolated human neutrophils were used promptly.

### Example 2

### Cell Culture and the Differentiation of HL60 Cells

U937 (human histiocytic lymphoma cells), HL60 (human promyelocytic leukemia cells), NIH3T3 (NIH Swiss mouse embryo fibroblasts), and PC12 (rat adrenal pheochromocytoma cells) were obtained from the American Type Culture Collection (Rockville, Md.) and HeLa (human adenocarcinoma cells) Tet-off cells were purchased from Clontech (Palo Alto, Calif., U.S.A.) and maintained as recommended. The cells were maintained at about 1×10⁶ cells/ml under standard incubator conditions (humidified atmosphere, 95% air, 5% CO₂, 37° C.). HL60 cells were induced to differentiate into the granulocyte phenotype by adding dimethylsulfoxide (DMSO) (final concentration 1.25% v/v) for 5 days, as has been described before (Ithoh et al., 1998).

### Example 3

### Establishment of Cell Lines

pRevTRE vector containing the cDNA of rat RGS2-GFP was transfected into Hela Tet-off cells using Lipofectamine. Selection was performed in DMEM supplemented with 2 μg/ml of tetracycline and 500 μg/ml of hygromycin B. Two weeks later, several well-isolated colonies were picked out and analyzed by western blotting to determine the expression level of RGS2 in the absence of tetracycline. For these experiments, HeLa cells were cultured for 48 h in the absence or in the presence of tetracycline.

### Example 4

### Measurement of Superoxide Anion Generation

Superoxide anion production was measured by monitoring chemiluminescence in the presence of the chemiluminogenic probe lucigenin (Bureau et al., 2001). Prepared neutrophils were plated in 96 wells and stimulated with chemicals at concentrations of 2.5, 5, 10, 15, 20, 25 and 50 μM, and then the lucigenin (40 μM) was added. Luminescence, measured with a Luminoskan (Labsystems, Inc., Helsinki, Finland), was integrated over 10 s intervals for a total of 3 min at room temperature.

### Example 5

### Measurement of [Ca²⁺]ᵢ

The chemically induced [Ca²⁺]ᵢ rise was measured using fura-2/AM (Grynkiewicz et al., 1985). Freshly prepared human neutrophils were incubated in serum free RPMI 1640 medium with 3 μM of fura-2/AM at 37° C. for 30 min with continuous stirring. After washing with serum free RPMI 1640 medium, the cells were suspended in serum free RPMI containing 250 μM of sulfinpyrazone to prevent dye leakage. About 2×10⁶ cells were suspended in Ca²⁺ free Locke's solution (158.4 mM NaCl, 5.6 mM KCl, 1.2 mM MgCl₂, 5 mM HEPES pH 7.3, 10 mM glucose, and 0.2 mM EGTA) for each measurement. Changes in the fluorescence ratio were measured at an emission wavelength of 500 nm for dual excitation wavelengths at 340 nm and 380 nm. The calibration of the fluorescence ratio versus [Ca²⁺]ᵢ was performed as described by Grynkiewicz (Grynkiewicz et al., 1985).

### Example 6

### Measurement of the Formation of Inositol Phosphates in Cells

The chemically induced formation of inositol phosphates was determined as described previously (Baek et al., 1996). Cells grown in culture were harvested by centrifugation, washed with inositol-free RPMI 1640 medium, and resuspended at a density of 2×10⁶ cells/ml in the same medium. The cells were then labeled with myo-[³H] inositol (1 μCi/10⁶ cells) for 24 h at 37° C. and rinsed twice with inositol-free RPMI 1640 medium containing 0.5% fetal bovine serum, 20 mM of Hepes at pH 7.2, 20 mM of LiCl, and bovine serum albumin (1 mg/ml) and then resuspended at a density of 2×10⁷ cells/ml. A portion (0.1 ml) of the cell suspension was transferred to a microcentrifuge tube and incubated at 37° C. for 15 min. PIP2 hydrolysis was initiated by adding chemicals or solvents for the indicated times. Reactions were terminated by adding 200 μl of ice-cold 10% perchloric acid (HClO₄). After 30 min in an ice bath, the tubes were centrifuged, and the supernatants were diluted 5-fold with distilled water and applied to Bio-Rad Dowex AG 1-X 8 anion exchange columns. Each column was then washed with 2 ml of distilled water and this was followed by 10 ml of 60 mM ammonium formate containing 5 mM sodium tetraborate. Total inositol phosphates were eluted with a solution containing 1 M ammonium formate and 0.1 M formic acid. The radioactivity of the [3H] inositol phosphates was determined using a scintillation counter (Tri-Packard, Meriden, Conn.).

### Example 7

### Measurement of Cyclic AMP Generation

To measure the cAMP level, we used a radio receptor assay (Pio et al., 2001), which was based on the competition between unlabeled cAMP (in the sample) and a fixed quantity of [³H] labeled cAMP, for a protein with a high cAMP specificity and affinity. The amount of labeled protein-cAMP complex formed was inversely related to the amount of unlabeled cAMP present in the assay sample. Prepared cells were treated with m-3M3FBS (25 μM) and histamine (100 μM), and then lysed with Tris-EDTA containing 50 μM Ro20-1724, a phosphodiesterase inhibitor. After centrifugation, the supernatant was collected and [³H] cAMP and protein were added. The protein bound cAMP was separated from the unbound nucleotide by adsorbing the free nucleotide on to coated charcoal, and centrifuging. An aliquot of the supernatant was then collected for liquid scintillation counting. The amount of unlabeled cAMP (in the sample) was calculated by measuring the protein-bound radioactivity.

### Example 8

### Measurement of Phosphoinositide Hydrolysis In Vitro

PLC activity was assayed using [³H] PtdIns-4,5-P₂ as a substrate (Min et al., 1993). PtdIns-4,5-P₂-hydrolyzing activity was measured with mixed phospholipid micelles containing 120 μM phosphatidylethanolamine, 30 μM PtdIns-4,5-P₂, and 1 μCi/ml [³H] PtdIns-4,5-P₂. The lipids, in chloroform, were dried under a stream of nitrogen gas, suspended in assay buffer (20 mM HEPES pH 7.0, 120 mM NaCl, 2 mM MgCl₂, 40 or 100 μM CaCl₂, and 1 mg/ml bovine albumin serum (Bayer, Leverkusen, Germany)), and sonicated. All proteins added to the reaction mixture were dialyzed overnight against the assay buffer. Incubation was performed for 10 min at 37° C. in a 200 μl reaction mixture containing lipid micelles (5 μM [3H] PtdIns-4,5-P₂, 20,000 cpm). The reaction was stopped by adding 2 ml of CHCl₃: CH₃OH: HCl (50: 50: 0.3, v/v/v). The inositol triphosphates were extracted with 0.5 ml of 1 N HCl, and radioactivities in the upper aqueous phase were measured.

Results

### Example 9

### Identification of a Synthetic Compound that Strongly Enhances Superoxide Generation in Human Neutrophils

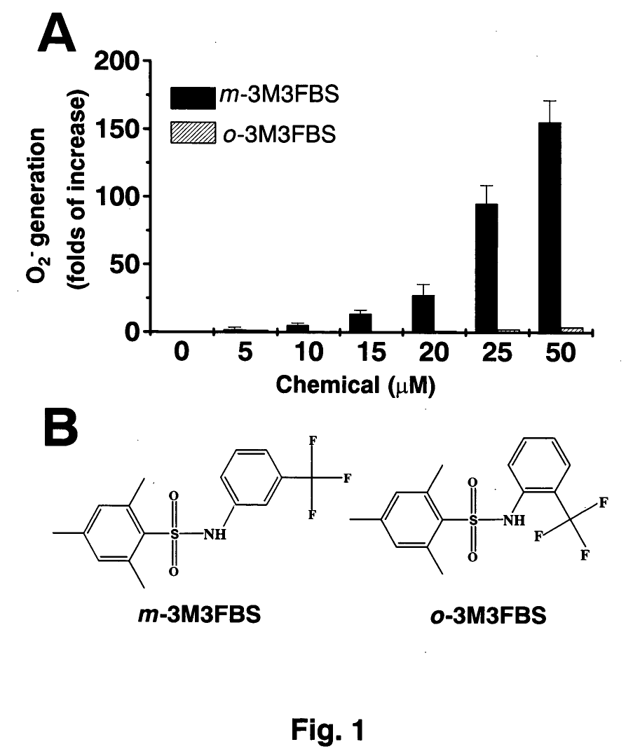

In this study, we screened around 10,000 chemicals in an effort to identify chemicals that stimulate superoxide generation in human neutrophils, and we found several chemicals that do so within the concentration range 20 μM -50 μM (data not shown). Among these, a chemical named m-3M3FBS proved to be the most potent in terms of its ability to stimulate the generation of superoxide. FIG. 1A shows that m-3M3FBS greatly enhanced superoxide generation within the concentration range 15 μM-50 μM. Interestingly, however, 2, 4, 6-trimethyl-N-(ortho 3-trifluoromethyl-phenyl)-benzenesulfonamide (o-3M3FBS), which has a similar structure to m-3M3FBS, except for the position of the trifloromethyl-phenyl group, did not affect superoxide generation up to 50 μM (FIG. 1A). Therefore, we used o-3M3FBS as an inactive analogue of m-3M3FBS. FIG. 1B shows the structures of m-3M3FBS and o-3M3FBS.

### Example 10

### m-3M3FBS Stimulates [Ca²⁺]ᵢ Increase in Neutrophils

Many extracellular agonists that stimulate superoxide anion generation in human phagocytic cells also increase [Ca²⁺]ᵢ (Bae et al., 1999; Liang et al., 1995). Therefore, we examined the effect of m-3M3FBS on [Ca²⁺]ᵢ in human neutrophils. As shown in FIG. 2A, m-3M3FBS caused a [Ca²⁺]ᵢ increase due to the transfer of calcium through the plasma membrane when Ca²⁺ levels were at physiologic levels extracellularly, whereas the inactive analogue, o-3M3FBS, failed to elicit this response. m-3M3FBS stimulated [Ca²⁺]ᵢ release in a concentration dependent manner showing the saturated maximal activity at 50 μM concentration (data not shown). Moreover, m-3M3FBS also evoked a [Ca²⁺]ᵢ increase in the extracellular calcium-depleted condition. These findings demonstrate that m-3M3FBS induces intracellular calcium increases as a result of both plasma membrane calcium entry and the release of intracellularly stored calcium.

Since intracellular calcium mobilization can be caused by the activation of PLC (Rhee, 2001), we undertook to prevent this compound response by blocking the activation of PLC with the membrane-permeable PLC inhibitor, U-73122. A 3-5-min pretreatment of neutrophils with U-73122 has been previously documented to fully prevent PLC activation upon agonist stimulation (Hershfinkel et al., 2001). As shown in FIG. 2B, pretreatment of neutrophils with 4 μM of U-73122 resulted in the complete inhibition of the calcium signal induced by m-3M3FBS. However, preincubating the same cells with U-73343, an inactive analogue of U-73122, had no effect on the m-3M3FBS-evoked response. This result indicates that [Ca²⁺]ᵢ increase induced by m-3M3FBS is mediated by PLC activity. Preincubation of neutrophils with U-73122 in the presence of extracellular calcium also completely blocked m-3M3FBS-induced calcium signal (data not shown). The results suggest that intracellular calcium increases due to plasma membrane calcium entry are mediated by intracellular calcium release.

### Example 11

### m-3M3FBS Stimulates the Formation of Inositol Phosphates in U937 Cells

Based on previous data, it was expected that m-3M3FBS would influence the activity of PLC. We next examined whether m-3M3FBS could stimulate PLC activation by measuring total inositol phosphate formation in U937 cells. After labeling with myo-[³H] inositol (1 μCi/10⁶ cells), the cells were treated with m-3M3FBS or o-3M3FBS. As shown in FIG. 3, the accumulation of inositol phosphates after treatment with m-3M3FBS increased gradually, giving a fold increase of 2.5 at 50 μM of m-3M3FBS. The concentration dependency of m-3M3FBS-induced inositol phosphates formation was closely correlated with that of m-3M3FBS-induced superoxide generation (FIG. 1A). In contrast, o-3M3FBS had no effect on PLC activity. This result indicates that m-3M3FBS stimulates PLC activation.

### Example 12

### m-3M3FBS has no Cell Type Specificity

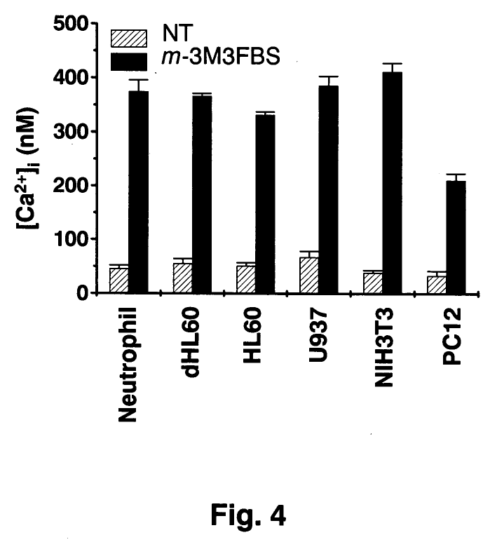

Up to this point, we had observed that m-3M3FBS caused superoxide anion generation in a PLC-dependent manner, and we wondered whether this chemical could affect signaling molecules upstream of PLC. In case of the PLCB series, generally ligand specific receptor and heterotrimeric G proteins are located upstream of signaling molecules (Rhee, 2001; Rhee and Bae, 1997). First, we investigated if m-3M3FBS has a specific receptor. As shown in FIG. 4, m-3M3FBS induced calcium increase in all cell lines (human neutrophils, HL60, differentiated HL60, U937, NIH 3T3, and PC12) examined, showing no cell type specificity.

### Example 13

### m-3M3FBS Desensitizes the Calcium Increase Induced by Other Agonists

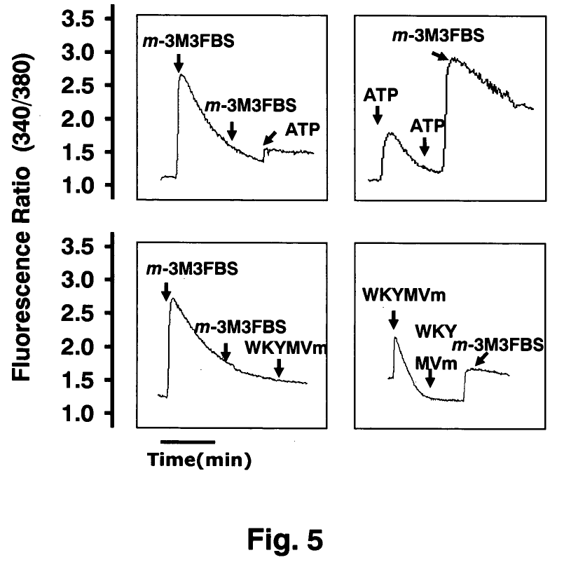

We next investigated the capacity of m-3M3FBS to desensitize other extracellular agonists, by examining its effect on ATP, and a synthetic leukocyte chemoattractant peptide Trp-Lys-Tyr-Met-Val-D-Met-CONH₂ (WKYMVm), which is known to stimulate PLC enzymes (Seo et al., 1997; Bae et al., 2000). In cross-desensitization experiments, stimulation of the cells with m-3M3FBS significantly reduced cellular responses to ATP and WKYMVm (FIG. 5). However, the administration of m-3M3FBS after stimulating cells with ATP or WKYMVm elicited still further [Ca²⁺]ᵢ increases (FIG. 5). Therefore, the m-3M3FBS-induced [Ca²⁺]ᵢ increase was more potent than that induced by other agonists, demonstrating that m-3M3FBS may more strongly affect the PLC enzyme(s) than other extracellular agonists. These results suggest that the target of m-3M3FBS is a common mediator of cell surface receptors, such as PLC or heterotrimeric G-protein.

### Example 14

### m-3M3FBS-Induced Signaling is not G-Protein-Dependent

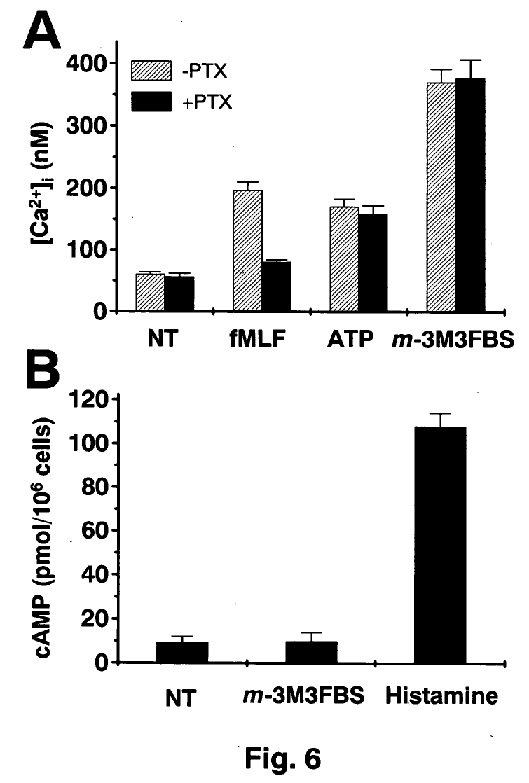

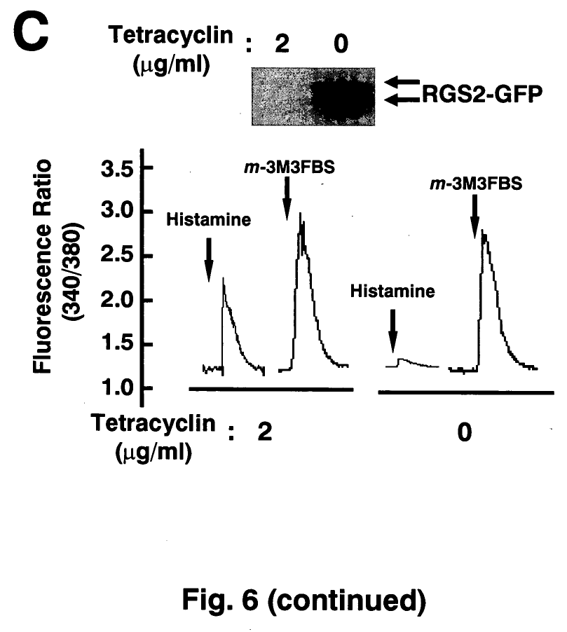

Several extracellular signals, including those due to many chemoattractants, activate phagocytic cells via pertussis toxin (PTX)-sensitive Gi-proteins (Feniger-Barish et al., 2000; Mellado et al., 2001). Therefore, we investigated the involvement of PTX-sensitive Gi-proteins upon m-3M3FBS-induced neutrophil activation. As shown in FIG. 6A, fMLF, a chemoattractant that signals through PTX-sensitive Gi-proteins (Jiang et al., 1996), induced [Ca²⁺]ᵢ increase and this response was inhibited by preincubating neutrophils with PTX (1 μg/ml) for 90 min. However, the calcium increase caused by m-3M3FBS was insensitive to PTX implying that Gi-proteins are not involved in m-3M3FBS-induced [Ca²⁺]ᵢ increase.

Adenylate cyclase integrates positive and negative signals that act through Gs protein-coupled cell-surface receptors to finely regulate levels of cAMP within several types of cells (Simonds, 1999). We next examined whether m-3M3FBS could activate Gs-proteins by measuring the change in the intracellular cAMP level. As shown in FIG. 6B, histamine is known to bind to Gs- and Gq-coupled receptors (Kilts et al, 2000; Kuhn et al., 1996), which potently induce cAMP production, but when m-3M3FBS was added to human neutrophils, it didn't change the cAMP level. These results suggest that m-3M3FBS cannot activate Gs-proteins.

The regulators of G protein signaling (RGS) proteins are GTPase activating proteins that inhibit signaling via heterotrimeric G proteins. Recently, it was reported that RGS2 is a selective inhibitor of Gq-protein function (Heximer et al., 1997). Therefore, we checked the involvement of Gq-proteins in m-3M3FBS-induced signaling using HeLa cells, which overexpress wild-type RGS2 under the control of an inducible tetracycline-regulated promoter. As shown in FIG. 6C, both histamine and m-3M3FBS caused [Ca²⁺]ᵢ increases in the presence of tetracycline. However, the calcium increases induced by histamine and m-3M3FBS were altered when RGS2 was overexpressed in cells, because whereas the histamine-induced calcium response was completely blocked by RGS2 induction, the m-3M3FBS-induced Ca²⁺ response was unaffected. These results suggest that Gq-proteins are not involved in the m-3M3FBS-mediated signaling pathway.

### Example 15

### m-3M3FBS Directly Activates PLC In Vitro

Since m-3M3FBS did not seem to act on cell surface receptor(s) or G-protein(s), we investigated the effect of m-3M3FBS on PLC activity directly. PLC activity was assayed using [3H] PtdIns-4,5-P₂ as substrate. Initially, we examined the activity of PLC β2, because it is highly expressed in immune cells (Bertagnolo et al., 2000). As shown in FIG. 7A, although m-3M3FBS had no effect at low doses (ca., 2.5 and 5 μM), the PIP₂-hydrolyzing activity of PLC β2 was enhanced at m-3M3FBS concentrations exceeding 10 μM. However, the inactive form of m-3M3FBS had no effect at this or at higher concentrations (FIG. 7A). We also tested the effect of m-3M3FBS on in vitro PLCβ2 activity using [³H] PtdIns as substrate and found that PLCβ2 activity was significantly increased by the compound showing similar pattern with the experiments using [³H] PtdIns-4,5-P₂ as substrate (data not shown).

We next tested several other purified PLC isozymes (beta3, gamma1, gamma2, and delta1) to check the isozyme specificity of m-3M3FBS. As shown in FIG. 7B, m-3M3FBS augmented the activity of all PLC isozymes tested, whereas o-3M3FBS did not affect PLC activity. These results indicate that m-3M3FBS elevates the PIP₂-hydrolyzing activity of PLC in vitro, without showing isotype specificity.

References

Bae Y S, Bae H, Kim Y, Lee T G, Suh P G and Ryu S H (2001) Identification of novel chemoattractant peptides for human leukocytes. Blood 97:2854-2862.

Bae Y S, Kim Y, Kim J H, Lee T G, Suh P G and Ryu S H (2000) Independent functioning of cytosolic phospholipase A2 and phospholipase D1 in Trp-Lys-Tyr-Met-Val-D-Met-induced superoxide generation in human monocytes. J Immunol 164:4089-4096.

Bae Y S, Ju S A, Kim J Y, Seo J K, Baek S H, Kwak J Y, Kim B S, Suh P G and Ryu S H (1999) Trp-Lys-Tyr-Met-Val-D-Met stimulates superoxide generation and killing of Staphylococcus aureus via phospholipase D activation in human monocytes. J Leukoc Biol 65:241-248.

Baek S H, Seo J K, Chae C B, Suh P G and Ryu S H (1996) Identification of the peptides that stimulate the phosphoinositide hydrolysis in lymphocyte cell lines from peptide libraries. J Biol Chem 271:8170-8175.

Bertagnolo V, Marchisio M, Pierpaoli S, Colamussi M L, Brugnoli F, Visani G, Zauli G and Capitani S (2002) Selective up-regulation of phospholipase C-beta2 during granulocytic differentiation of normal and leukemic hematopoietic progenitors. J Leukoc Biol 2002 71:957-965.

Bureau C, Bernad J, Chaouche N, Orfila C, Beraud M, Gonindard C, Alric L, Vinel J P and Pipy B (2001) Nonstructural 3 protein of hepatitis C virus triggers an oxidative burst in human monocytes via activation of NADPH oxidase. J Biol Chem 276:23077-23083.

Feniger-Barish R, Belkin D, Zaslaver A, Gal S, Dori M, Ran M and Ben-Baruch A (2000) GCP-2-induced internalization of IL-8 receptors: hierarchical relationships between GCP-2 and other ELR(+)-CXC chemokines and mechanisms regulating CXCR2 internalization and recycling. Blood 95:1551-1159.

Grynkiewicz G, Poenie M and Tsien R Y (1985) A new generation of Ca2+ indicators with greatly improved fluorescence properties. J Biol Chem 260:3440-3450.

Hershfinkel M, Moran A, Grossman N and Sekler I (2001) A zinc-sensing receptor triggers the release of intracellular Ca2+ and regulates ion transport. Proc Natl Acad Sci USA 98:11749-11754.

Heximer S P, Watson N, Linder M E, Blumer K J and Hepler J R (1997) RGS2/GOS8 is a selective inhibitor of Gqalpha function. Proc Natl Acad Sci USA 94:14389-14393.

Horstman D A, DeStefano K and Carpenter G (1996) Enhanced phospholipase C-gammal activity produced by association of independently expressed X and Y domain polypeptides. Proc Natl Acad Sci USA 93:7518-7521.

Itoh K, Okubo K, Utiyama H, Hirano T, Yoshii J and Matsubara K (1998) Expression profile of active genes in granulocytes. Blood 92:1432-1441.

Jiang H, Kuang Y, Wu Y, Smrcka A, Simon M I and Wu D (1996) Pertussis toxin-sensitive activation of phospholipase C by the C5a and fMet-Leu-Phe receptors. J Biol Chem 271:13430-13434.

Kilts J D, Gerhardt M A, Richardson M D, Sreeram G, Mackensen G B, Grocott H P, White W D, Davis R D, Newman M F, Reves J G, Schwinn D A and Kwatra M M (2000) Beta(2)-adrenergic and several other G protein-coupled receptors in human atrial membranes activate both G(s) and G(i). Circ Res 87:705-709.

Kim Y H, Park T J, Lee Y H, Baek K J, Suh P G, Ryu S H and Kim K T (1999) Phospholipase C-delta1 is activated by, capacitative calcium entry that follows phospholipase C-beta activation upon bradykinin stimulation. J Biol Chem 274:26127-26134.

Kuhn B, Schmid A, Harteneck C, Gudermann T and Schultz G (1996) G proteins of the Gq family couple the H2 histamine receptor to phospholipase C. Mol Endocrinol 10:1697-1707.

Liang L and Huang C K (1995) Activation of multiple protein kinases induced by cross-linking of Fc gamma RII in human neutrophils. J Leukoc Biol 57:326-331.

McLaughlin A P and De Vries G W (2001) Role of PLCgamma and Ca(2+) in VEGF- and FGF-induced choroidal endothelial cell proliferation. Am J Physiol Cell Physiol 281:C1448-C1456.

Mellado M, Rodriguez-Frade J M, Vila-Coro A J, Fernandez S, Martin de Ana A, Jones D R, Toran J L and Martinez-A C (2001) Chemokine receptor homo- or heterodimerization activates distinct signaling pathways. EMBO J 20:2497-2507.

Min D S, Kim D M, Lee Y H, Seo J, Suh P G and Ryu S H (1993) Purification of a novel phospholipase C isozyme from bovine cerebellum. J Biol Chem 268:12207-12212.

Noh D Y, Shin S H and Rhee S G (1995) Phosphoinositide-specific phospholipase C and mitogenic signaling. Biochim BiophysActa 1242:99-113.

Pio R, Martinez A, Unsworth E J, Kowalak J A, Bengoechea J A, Zipfel P F, Elsasser T H and Cuttitta F (2001) Complement factor H is a serum-binding protein for adrenomedullin, and the resulting complex modulates the bioactivities of both partners. J Biol Chem 276:12292-12300.

Rhee S G (2001) Regulation of phosphoinositide-specific phospholipase C. Annu Rev Biochem 70:281-312.

Rhee S G and Bae Y S (1997) Regulation of phosphoinositide-specific phospholipase C isozymes. J Biol Chem 272:15045-15048

Rosania G R, Merlie J Jr, Gray N, Chang Y T, Schultz P G and Heald R (1999) A cyclin-dependent kinase inhibitor inducing cancer cell differentiation: biochemical identification using Xenopus egg extracts. Proc Natl Acad Sci U S A 96:4797-4802.

Seo J K, Choi S Y, Kim Y, Baek S H, Kim K T, Chae C B, Lambeth J D, Suh P G and Ryu S H (1997) A peptide with unique receptor specificity: stimulation of phosphoinositide hydrolysis and induction of superoxide generation in human neutrophils. J Immunol 158:1895-1901.

Simonds W F (1999) G protein regulation of adenylate cyclase. Trends Pharmacol Sci 20:66-73.

Strizki J M, Xu S, Wagner N E, Wojcik L, Liu J, Hou Y, Endres M, Palani A, Shapiro S, Clader J W, Greenlee W J, Tagat J R, McCombie S, Cox K, Fawzi A B, Chou C C, Pugliese-Sivo C, Davies L, Moreno M E, Ho D D, Trkola A, Stoddart C A, Moore J P, Reyes G R and Baroudy B M (2001) SCH-C (SCH 351125), an orally bioavailable, small molecule antagonist of the chemokine receptor CCR5, is a potent inhibitor of HIV-1 infection in vitro and in vivo. Proc Natl Acad Sci U S A 98:12718-12723.

Tian S S, Lamb P, King A G, Miller S G, Kessler L, Luengo J I, Averill L, Johnson R K, Gleason J G, Pelus L M, Dillon S B and Rosen J (1998) A small, nonpeptidyl mimic of granulocyte-colony-stimulating factor Science 281:257-259.

Zhang B, Salituro G, Szalkowski D, Li Z, Zhang Y, Royo I, Vilella D, Diez M T, Pelaez F, Ruby C, Kendall R L, Mao X, Griffin P, Calaycay J, Zierath J R, Heck J V, Smith R G and Moller D E (1999) Discovery of a small molecule insulin mimetic with antidiabetic activity in mice. Science 284:974-977.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method for activating phospholipase C (PLC) activity in a cell comprising contacting a cell comprising phospholipase C with compound of Formula I: 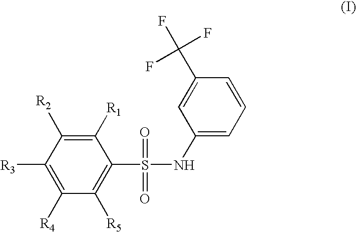
wherein
R₁ to R₅ are independently selected from H, OH group, a C₁-C₄ alkyl group, a halogen, an OR₆ group, COOH, COOR₇, an alkylene ester group according to the structure-(CH₂)ₘCOOR₇ or a CF₃ group;
R₆ is a C₁-C₄ alkyl group (preferably, C₁-C₄ alkyl), or a COR₇ group; R₇ is a C₁-C₂₀ alkyl group, preferably a C₁-C₄ alkyl group.

2. The method according to claim 1, wherein the compound of Formula I is
4-tert-Butyl-N-(3-trifluoromethyl-phenyl)-benzenesulfonamide, 3,5-Di-tert-butyl-4-fluoro-N-(3-trifluoromethyl-phenyl)benzenesulfonamide,
or 2,4,6-Trimethyl-N-(meta-3-trifluoromethyl-phenyl)-benzenesulfonamide (m-3M3FBS).

3. The method according to claim 2, wherein the compound of Formula I is 2,4,6-Trimethyl-N-(meta 3-trifluoromethyl-phenyl)-benzenesulfonamide (m-3M3FBS).

4. The method of claim 1, wherein the PLC comprises isoforms beta, gamma and delta.

5. The method of claim 1, wherein the compound of Formula I does not inhibit heterotrimeric G proteins, PI 3-kinase or phospholipase D.

6. The method of claim 1, wherein the PLC activity is production of superoxide.

7. The method of claim 1, wherein the PLC activity is increasing intracellular level of calcium.

8. The method of claim 1, wherein the PLC activity is stimulating formation of inositol phosphate formation in a cell.

9. The method according to claim 1, wherein the cell is a neutrophil.

10. The method according to claim 1, wherein the cell is a neuronal cell.

11. The method according to claim 1, wherein the cell is a fibroblast cell.

12. The method according to claim 1, comprising contacting the cell with an inactive control compound and comparing results of the compound of Formula I and the control compound.

13. The method according to claim 12, wherein the control compound is 2,4,6-Trimethyl-N-(ortho 3-trifluoromethyl-phenyl)-benzenesulfonamide (o-3M3FBS).

14. A method for screening for candidate antitumor agent comprising contacting a cell expressing phospholipase C with the compound of Formula I to activate the phospholipase C activity, and then contacting the cell with a compound to determine whether phospholipase C activity is inhibited, wherein inhibition of phospholipase C activity indicates that the compound is an anti-tumor candidate compound.

15. A method for treating a brain disorder in a subject comprising administering to a subject thereof therapeutically effective amount of the compound of Formula I.

16. Instructions comprising the method according to claim 1.

17. The instructions according to claim 16, which is a web page, catalog or an experimental manual.

18. A method for controlling PLC-mediated cell signaling comprising contacting a cell comprising phospholipase C with controlled amounts of the compound of Formula I.

19. A method for increasing bactericidal activity in a subject comprising administering to the subject an effective amount of the compound of Formula I.
